(19) United States
(12) Patent Application Publication
(10) Pub. No.: US 2015/0119866 A1
(43) Pub. Date: Apr. 30, 2015

(54) Surgical Marker and Cap

(71) Applicant: Igor Nichiporenko, Miami Beach, FL (US)

(72) Inventor: Igor Nichiporenko, Miami Beach, FL (US)

(21) Appl. No.: 14/065,958

(22) Filed: Oct. 29, 2013

(51) Int. Cl. A61B 19/00

(52) U.S. Cl. CPC A61B 19/44
USPC 606/1

(57) ABSTRACT

A surgical marker and cap are useable to mark tissue in laparoscopic procedures. The surgical marker connects to a rod and can be pushed through a cannula to target tissue that is to be marked. The marker can include a decreased length by moving the location of the ink reservoir from the location in a typical marker to a location in the cap. The marker includes a connector to allow it to be easily connected to a rod that contains a peg. The marker is usable in laparoscopic procedures such as the connection of the leads of a diaphragm pacemaker.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to surgical markers.

2. Description of the Related Art

Laparoscopic surgery, also called minimally invasive surgery (MIS), bandaid surgery, or keyhole surgery, is a surgical technique in which operations in the abdomen are performed through small incisions (usually 0.5-1.5 cm) as opposed to the larger incisions needed in traditional laparotomy.

Laparoscopic surgery provides a number of advantages to the patient versus an open procedure. These include reduced pain due to smaller incisions and hemorrhaging, and shorter recovery time.

A disadvantage of laparoscopic surgery is that the surgeon cannot see the targeted tissue directly. Several tools and techniques enable surgeons to locate the targeted tissue.

One such device is an endoscope. An endoscope is an instrument used to examine the interior of a hollow organ or cavity of the body. The endoscope is inserted through the small incision and delivered to the tissue that is to be observed. An endoscope typically includes a rigid or flexible tube, a light delivery system to illuminate the organ or object under inspection, a lens system transmitting the image from the objective lens to the viewer, an eyepiece, and an additional channel to allow entry of medical instruments or manipulators.

An example of a laparoscopic procedure is the connection of pacemaker leads during insertion of a diaphragmatic pacemaker. A diaphragmatic pacemaker, in medicine, is a surgically implanted device used to help patients breathe following complications from spinal cord injuries. The device works through pacing of the diaphragm. Diaphragm pacing originally required a surgical opening of the chest cavity (thoracotomy) to implant the electrodes. It is now done by laparoscopy through small openings in the abdominal cavity. Patients undergo laparoscopic implantation of electrodes in the muscle of the diaphragm and initial electrical stimulation.

An endoscope is used to observe the nerves on the diaphragm. Next, the intended connection points of the leads on the nerves are mapped onto a monitor displaying an image being recorded and transmitted by the endoscope. The image on the display is a live image that is continuously updated. However, the mapped points are only mapped on the display, and not on the anatomy. As the patient and or endoscope move with respect to each other, the mapped points become less and less accurate.

To mark tissue during laparoscopic procedures, make-shift markers have been improvised. Typically, a surgical marking pen is disassembled by the surgeon to remove the nib of the marker. The nib is held by an existing surgical grabber. The grabber with the nib is delivered to the target tissue and the tissue is marked. The nib often dislodges from the grabber. The nib can dislodge during delivery or retraction through the cannula. The nib can dislodge when it contacts tissue in the body. The nib can dislodge if it snags the edge of the cannula during retraction. If the nib is dislodged within a patient, the nib is very difficult to locate. The surgeon is faced with two undesirable alternatives: leaving the nib within the patient or enlarging the incision to search the patient more closely.

Most laparoscopic surgical product lines include a blunt dissector. Blunt dissection, as opposed to sharp dissection, involves the use of a blunt surface to break through the tissue, thereby preventing the damage and bleeding caused by lasers and scalpels, the tools of sharp dissection. Hard surgical sponges, generally known as peanuts or Kittner sponges, or a surgeon's fingers are often used as blunt dissectors. A peanut is a tightly wound ball of absorbent material, such as gauze or other woven cotton, which typically is gripped with forceps. The weave of the material acts to abrade the tissue being dissected so that the dissection can be performed by either pulling on the tissue or by forcing the peanut through the tissue. The peanut is usually placed on a peg that is located at the distal end of a rod.

In light of the existing lines of surgical tools, a need exists to provide a surgical marker that can be connected to rods in existing lines of surgical products.

Phillips, US 2006/0167471, teaches a surgical marker. No detail is provided regarding the connection of the nib to the rod. In addition, the ink in the nib of the marker has significant risk of drying because the nib is so small and is not connected to a reservoir.

Accordingly, there is a need for a device that can mark tissue in laparoscopic procedures without risking leaving debris within a patient. In addition, such a device should work with existing lines of surgical products. Furthermore, the marker should be capable of being stored for typical times without risking of the ink in the marker drying.

### BRIEF SUMMARY OF THE INVENTION

An object of the invention is to provide a combined marker and dissector that overcomes the disadvantages of the devices of this general type and of the prior art.

With the foregoing and other objects in view there is provided, in accordance with the invention, a surgical marker for marking tissue laparoscopically. The marker includes a connector and a nib. The connector has a socket formed therein for receiving a peg of a rod. Once connected, the rod is used to push and/or retract the marker from with the body of a patient via a cannula. The rod can be used to maneuver the marker to tissue that is to be marked for subsequent procedure. Once the tissue is marked, the marker can be removed from the patient by retracting the rod. The nib of the marker is configured to hold ink and dispense the ink onto tissue. The nib is connected to the proximal connector. The nib can be located distal to the connector so that the tip of the nib leads as the marker is pushed through the cannula. The nib should be secured to the connector to guarantee that the nib does not separate from the connector.

The marker can be no wider than the rod. A typical rod has a diameter of 0.5 cm wide. In addition, the marker can have a smooth edge so that it does not get snagged during insertion or removal via a cannula. The marker and the connector can have a smooth connection when joined so that the marker and/or the rod do not get snagged during deployment or retraction.

The nib can be made from made of porous, pressed fibers such as felt or synthetic fibers. The nib holds ink and dispenses the ink onto a surface when the nib contacts the surface. Both the nib and the ink can be sterilized and kept clean during manufacture. The ink should be in a color that contrasts tissue to allow it to be easily seen via a laparoscope, for example, blue.

The marker can include a holder for securing the nib to the connector. The holder can have a hole formed therein. The holder can be configured to be placed over the nib to allow a tip of the nib to extend from the hole when the holder is fastened to the connector. The holder can fit over the connector to form a snug connection. A snug connection is defined as one that can be twisted by hand.

An object of the invention is to provide a marker that is no wider than the rod used to insert and remove the marker. To meet this object, the connector can be configured to have a proximal wider portion and a distal narrower portion. An abutment is defined between the proximal wider portion and the distal narrower portion, where the diameter of the connector decreases. The holder has a proximal opening that can snugly fit over the narrower portion of the connector. The holder is slid onto the narrower portion until the proximal face of the holder contacts the abutment of the connector. The width of the abutment can be equal in size to the thickness of the holder wall. In this way, the outer surface of the holder is flush with the wider diameter of the connector when the holder is placed on the connector.

The holder can work to sandwich the nib between the connector and the holder to hold the nib in place.

The holder can include a passage from its proximal end to the hole in the holder, which is formed in the distal end of the holder. The passage can have a frustoconical shape to create a form locking connection with the nib to hold the nib within the holder but still allow the tip of the nib to emerge from the tip.

As stated, the connector has a proximal socket formed in the connector. The proximal socket has a length, i.e. a depth, that is at least as long as a length of the peg of the rod. The socket can have a diameter that is at least as wide as a diameter of the peg. The socket and peg should be sized so that the socket and the beg form a snug fit when the peg of the rod is inserted in the connector. The peg can have a cylindrical shape and the socket have a complimentary cylindrical shape.

The marker according to the invention can be combined with a rod for inserting and removing the marker into a patient via a cannula. The rod can have a distal peg. The peg is inserted in the socket of the connector. Once the marker is delivered to the target, the rod can be used to manipulate the marker to deposit ink on the target tissue.

In accordance with further objects of the invention, a cap for storing ink of a marker, in particular, a surgical marker is provided. An object of the invention is to provide a small marker that is stored with a large reservoir of ink to guarantee that the marker is sufficiently inked when used. To meet this object, a cap with an ink reservoir is provided. In this way, the marker can save the space that normally is taken by an ink reservoir that is proximally located relative to the tip of the marker.

The cap includes an enclosure. The enclosure can have many shapes although a generally cylindrically shaped can be provided. The enclosure has an opening formed therein. The opening is configured to receive the distal end of a marker. When the enclosure receives the marker, the size of the opening should be such that the enclosure and the marker form an air-tight closure. The air-tight closure prevents the ink on the nib of the marker from drying.

The cover includes an absorbent fiber block for holding ink. The absorbent fiber block is disposed in the enclosure. The block contacts the distal tip of the nib of the marker when the enclosure receives the marker. When the nib is in contact with the absorbent fiber block, ink stored in the absorbent fiber block can diffuse to the nib of the marker.

The absorbent fiber block can have a dimple formed therein. The dimple is configured to receive a tip of a nib of the marker when the enclosure receives the marker. When the tip is seated in the dimple, the amount of surface area of the nib and the absorbent fiber block that are in contact with each other is increased. The increased surface area increase diffusion of ink and limits evaporation of ink from the nib.

To secure the absorbent fiber block within the cap, the opening of the enclosure can include an inner proximal abutment. That is, the proximal abutment is proximal to the absorbent fiber block. The inner proximal abutment narrows the opening in the cap to a width that is narrower than a width of the absorbent fiber block. A distal plug can be placed on the cap. The absorbent fiber block is inserted via the distal end of the cap. Once inserted, the distal plug is placed on the cap to close the distal end of the cap and to secure the absorbent fiber block between the cap and the inner proximal abutment.

In accordance with a further object of the invention, a method for assembling a surgical marker is encompassed within the invention. A first step of the method involves providing a surgical marker according to the invention. The next involves providing a rod having a distal peg. The next involves inserting the distal peg into the socket of the connector.

In accordance with further objects of the invention, a method for marking tissue in a laparoscopic procedure is provided. The surgical marker according to the invention can be used in the procedure. The procedure includes the following steps. The first step is observing a target location in the patient with a laparoscope. The next step is connecting the marker to a rod by placing a peg on the rod in the socket formed in the connector of the marker. The next step is inserting a surgical marker to the target location by pushing the surgical marker with a rod through a cannula. The next step is identifying the target tissue by viewing the target tissue with the laparoscope. The marker can be manipulated and maneuvered using the rod while the surgeon views the tip of the market and the target tissue through the laparoscope. Additional steps in the procedure are possible.

The general procedure can be applied to connect pacemaker leads. In such a case, the targeted tissue is the nerve locations on the patient's diaphragm. The targeted tissue is marked with the marker. Once marked, a pacemaker lead is connected to the marked target tissue while observing said marked target tissue with said laparoscope.

Other features that are considered as characteristic for the invention are set forth in the appended claims.

Although the invention is illustrated and described herein as embodied in a combined marker and dissector, the invention should not be limited to the details shown in those embodiments because various modifications and structural changes may be made without departing from the spirit of the invention while remaining within the scope and range of equivalents of the claims.

The construction and method of operation of the invention and additional objects and advantages of the invention is best understood from the following description of specific embodiments when read in connection with the accompanying drawings.

### BRIEF DESCRIPTION OF THE SEVERAL VIEWS OF THE DRAWING

FIG. 1 is a diagrammatic, perspective view of a surgical marker according to the invention.

FIG. 2 is a diagrammatic, perspective, partial view of the surgical marker shown in FIG. 1 with a first embodiment of a cap shown in a detached position.

FIG. 3 is a diagrammatic, exploded, perspective view of the surgical marker shown in FIG. 2.

FIG. 4 is a diagrammatic, exploded, perspective view of a second embodiment of a cap.

FIG. 5 is a diagrammatic, exploded, partial, perspective view of the surgical marker shown in FIG. 1.

FIG. 6 is a diagrammatic sectional view of the second embodiment of the cap shown in FIG. 4 that includes a marker.

FIG. 7 is a side view of a third embodiment of the cap according to the invention.

FIG. 8 is a side sectional view of the second embodiment of the marker shown in FIG. 1 that inserted in the second embodiment of the cap shown in FIG. 7.

FIG. 9 is an exploded, partial, side-sectional view of the marker and cap shown in FIG. 8.

FIG. 10 is an exploded, side view of the marker and cap shown in FIG. 8.

FIG. 11 is an exploded, side sectional view of the marker and cap shown in FIG. 8.

### DETAILED DESCRIPTION OF THE INVENTION

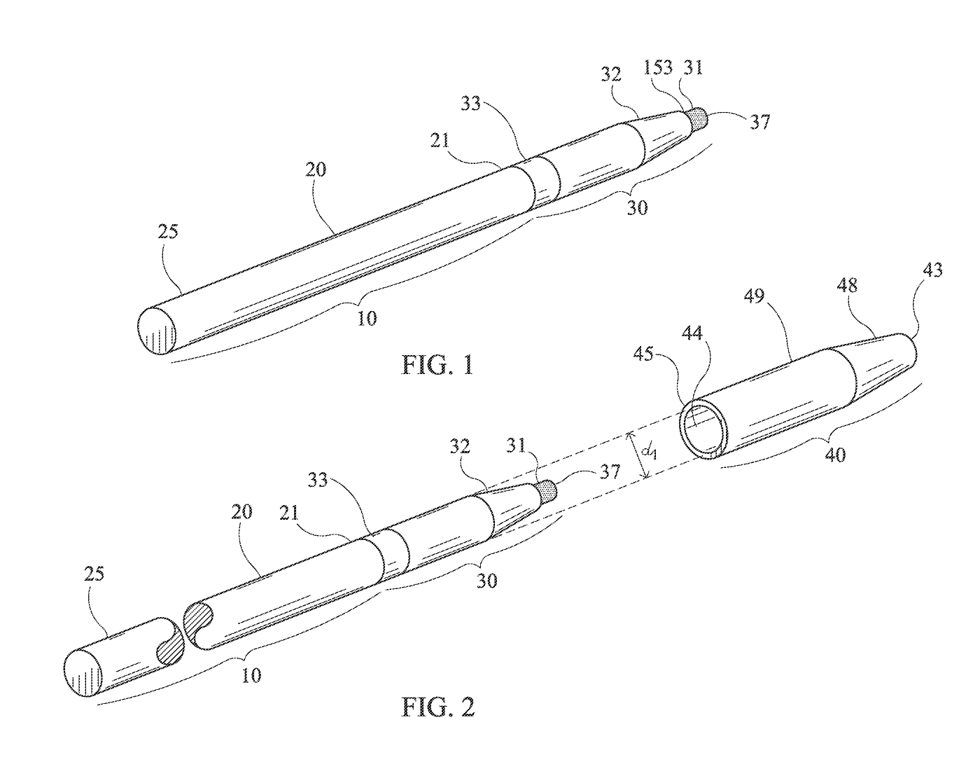

FIG. 1 shows an embodiment of a surgical marker 30. The surgical marker 30 may be referred to as a “marker”. While the preferred embodiment of a method of using the surgical marker 30 is for surgery, other procedures than need to mark a target at a distance from the user might use the surgical marker 30. The surgical marker 30 is particularly useful in procedures in which the user needs to mark a target via conduit or cannula. A rod assembly 10 is used to deliver the surgical marker 30 via a cannula. The cannula is not shown. Examples of compatible cannulas are those that are sold under the trade name COVIDIEN.

The rod assembly 10 includes a rod 20. The rod 20 is cylindrical. The rod 20 is made of a flexible resilient material. The rod 20 has a diameter that is less than an inner diameter of the cannula. The rod 20 is configured to be pushed through the cannula to a targeted tissue within a patient's body. A preferred embodiment of the rod 20 has a diameter of five millimeters (5 mm); other widths are possible for different uses. In the drawings, the rod 10 is not necessarily drawn to scale. In particular, the length in proportion to the width could be different. A particularly, preferred embodiment of the rod 20 is a rod that is similar to a blunt dissector with the dissector removed such as the dissectors sold under the trade name ENDO PEANUT by Covidien. The rod 20 has a proximal end 25 and a distal end 21. The proximal end 25 is handled by the surgeon and is used to push the rod assembly 10 through the cannula. The distal end 21 ends in a peg 22. The peg 22 is a cylinder with smaller diameter than the rod 20.

The marker 30 includes a connector 33, a holder 32, and a nib 31. The connector 33 is used to connect the marker 30 to the rod assembly 10. The holder 32 encloses part of the nib 31. The holder 32 fastens to the connector 33 and secures the nib 31 to the connector 33. The holder 32 has an opening 153 formed in a distal end of the holder 32. The distal tip 37 of the nib 31 extends beyond the holder 32. The tip 37 of the nib 31 is used to write on a target, for example, targeted tissue.

FIG. 2 shows the surgical marker 30 and rod assembly 10 with a first embodiment of a cap 40. The cap 40 is an enclosure with a proximal opening for receiving the tip 37 of the marker 30. The cap 40 has a proximal cylindrical portion 49 and a distal frustoconical portion 48. The distal end 43 of the cap 40 is closed. The cap 40 has an inner tube wall 44. The rod diameter d₁ is approximately equal to a diameter of the opening 42. The diameter d₁ of the opening in relation to the diameter of the marker should be close enough to allow the cap 40 to receive the surgical mark 30 and form an airtight connection when inserted but still allow the cap 40 to be removed by hand from the surgical marker 30.

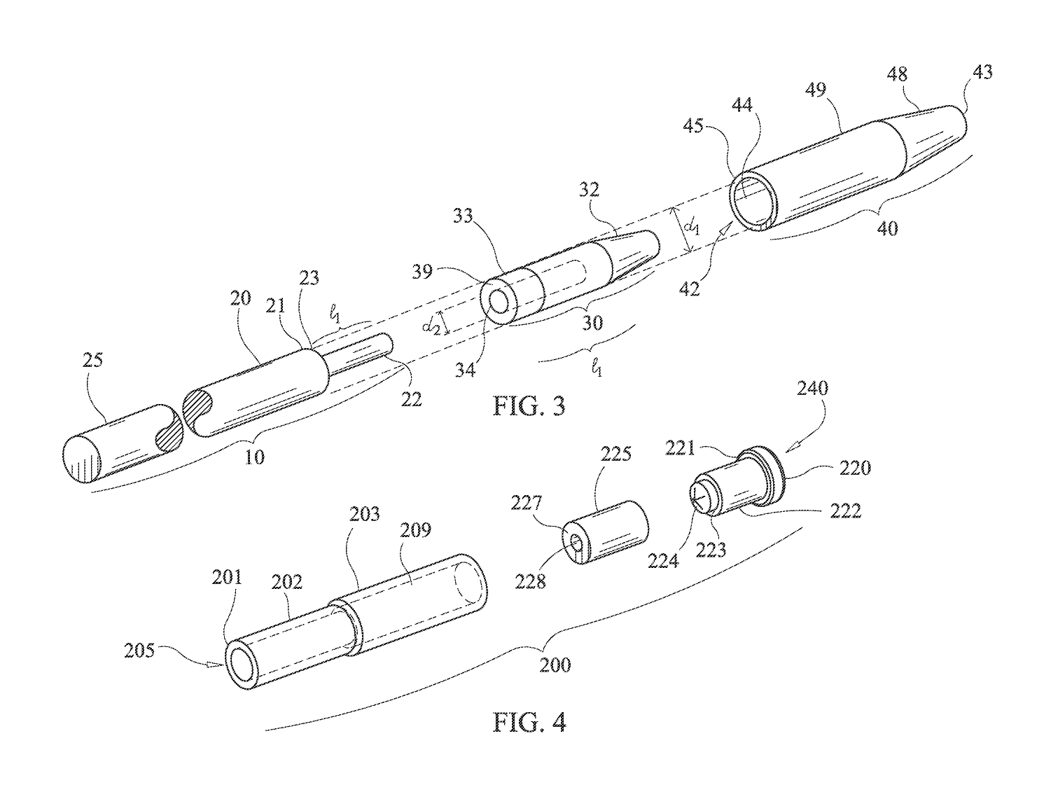

FIG. 3 shows an exploded view of the rod assembly 10, surgical marker 30, and cap 40 that is shown in FIG. 2. In FIG. 3, the nib 31 is not shown. A peg 22 is disposed on a distal face 23 of the rod 30. In the preferred embodiment, the peg 22 is cylindrical. The peg 22 has a length l₁ and a diameter d₂. The connector 33 of the marker 30 has a proximal abutment 39. A socket 34 is formed in the connector 33. The socket 34 has a diameter d₂ and a length l₂. The length l₂ of the socket 34 is at least as the length l₁ of the peg 22. The peg 22 inserts in the socket 34 to connect the rod assembly 10 and the marker 30. The diameters d₂ of the socket 34 and the peg 22 should be substantially equal such that a snug connection is formed such that more force than can be applied by hand is required to separate the rod assembly 10 from the marker 30.

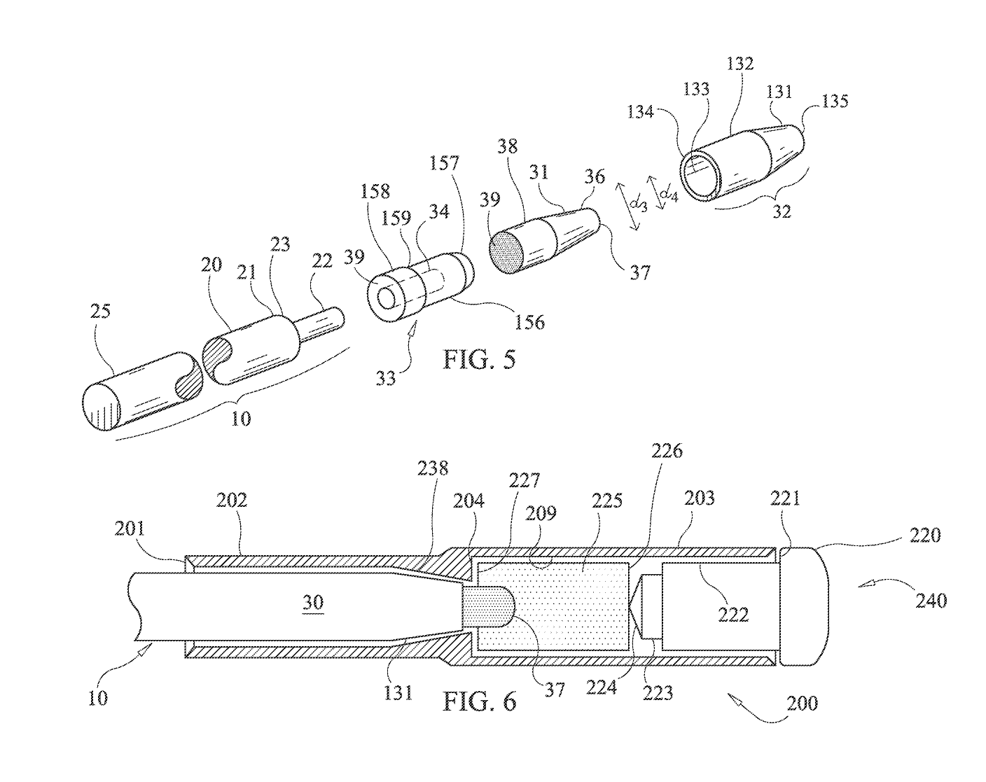

FIG. 5 shows a view of the embodiment in FIGS. 1-3 and details how the holder 32 secures the nib 31. The nib 31 is a block of absorbent fiber material. Synthetic fiber materials are preferred for sanitary reasons over natural fibers such as felt. The nib 31 holds and dispenses ink. The ink is preferably a highly visible color that contrasts the color of the tissue to be marked. Blue is typically a preferred color of ink. The nib 31 includes distal frustoconical portion 36 that has a distal tip 37. The nib 31 includes a proximal cylindrical portion 38 that has a proximal abutment 39.

FIG. 5 shows details of the connector 33. The connector includes a proximal wide cylindrical portion 158 and a distal narrow cylindrical portion 156. The wide cylindrical portion 158 has a diameter d₃. The narrow cylindrical portion 156 has a diameter d₄. An abutment 159 is defined between the wide cylindrical portion 158 and the distal narrow cylindrical portion 156. A frustoconical portion 157 is at the distal-most location of the connector 33 and provides a surface on which the nib 31 can rest.

FIG. 5 shows details of the holder 32. The holder 32 includes a distal frustoconical portion 131 and a proximal cylindrical portion 132. The holder 32 has an inner wall 133. Both ends of the holder 32 are open. A proximal face 134 is located on the cylindrical portion 132. A hole 135 is formed at the distal end of the frustoconical portion 131. The cylindrical portion 132, which is hollow has an outside diameter d₃ and an inside diameter d₄.

As shown in FIGS. 1, 2, and 5, when the holder 32 is fitted over the nib 31 and seated on the narrow cylindrical portion 156, the nib 31 is secured and the tip 37 of the nib 31 extends beyond the hole 135. The holder 32 should form a snug connection, which requires more torque to unscrew than can be applied by hand, when the holder is seated on the narrow cylindrical portion 156 because the inner diameter of the holder 32 and the outer diameter of the narrow cylindrical portion are equal d₄. The holder 32 and the wide cylindrical wall provide a smooth outer surface when the holder 32 is seated on the narrow cylindrical portion 156 because the outer diameter of the holder 32 and the outer diameter of the wide cylindrical wall 158 are equal d₃. The proximal face 134 of the holder 32 abuts the abutment 159 of the connector 33 when the holder 32 is seated on the connector 33.

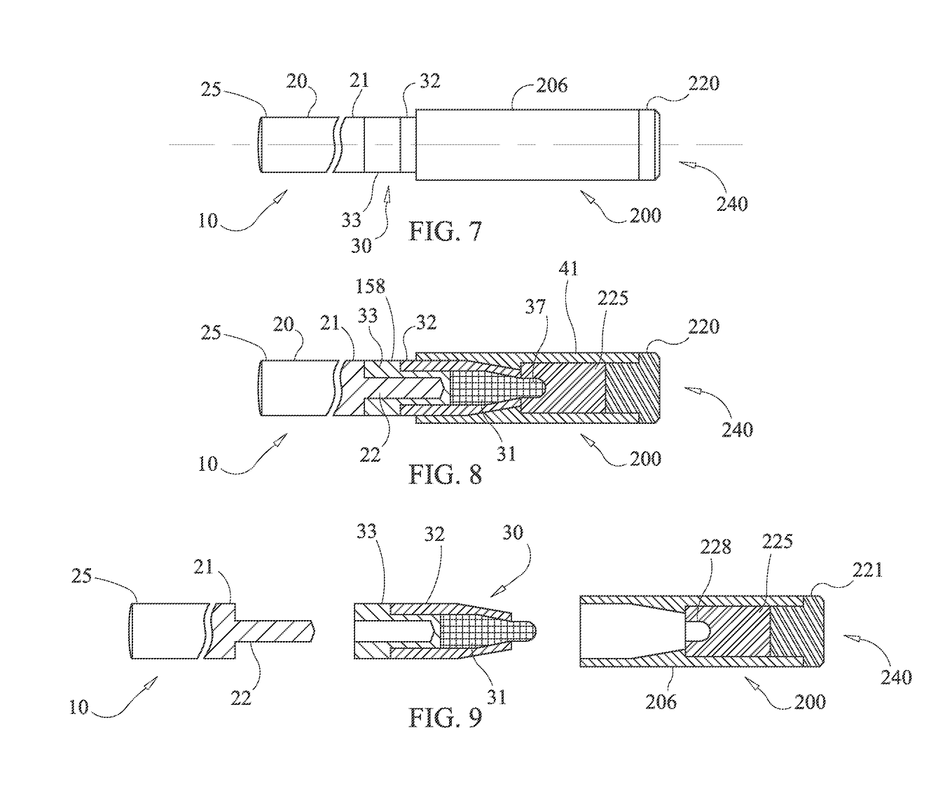

FIGS. 4 and 6 show a second embodiment of a cap 200 according to the invention. FIGS. 7-11 show a third embodiment of a cap. The cap 200 has an ink reservoir for wetting the nib of markers.

FIG. 6 shows the second embodiment of the cap 200 according to the invention. An enclosure for a marker 30 is formed by a generally cylindrical body that is capped at a distal end and open at a proximal end. A cylindrical, narrow wall 202 is provided at a proximal end of the cap 200. An inner diameter of the narrow wall 202 is sized to fit over an outer diameter of the marker 30. The narrow wall 202 has an opening 205 for receiving the distal end of the marker 30 as shown in FIG. 4. A flange 201 is provided to form an air-right fit when the marker 30 is inserted in the opening 205 of the cap.

The cap 200 includes an absorbent fiber block 225. The absorbent fiber block 225 is soaked with ink. The absorbent fiber block 225 contacts the nib 31 of the marker 30 when the marker 30 is inserted in the cap 200. Ink diffuses from the absorbent fiber block 225 into the nib 31 to keep the nip 31 moist and inked. As shown in FIG. 11, the absorbent fiber block 225 has a dimple 228 formed in the absorbent fiber block 225. The dimple 228 is shaped to complement the shape of the tip 37 of the nib 31. As shown in FIG. 6, the tip 37 of the nib seats in the dimple 228 when the marker 30 is fully inserted in the cap 200.

FIG. 6 shows that the narrow wall 202 has a frustoconical portion 238. The frustoconical portion 131 of the holder 32 complements the frustoconical portion 238 of the narrow wall 202 and abuts the frustoconical portion 238 when the marker 30 is fully inserted in the cap 200.

FIG. 6 shows the wide outer wall 203 and the inner wall 209. A proximal abutment 204 is defined between where the cap reduces inner diameter between the wide outer wall 203 and the narrow wall 202. A proximal face 227 of the absorbent fiber block 225 abuts the proximal abutment 204. The proximal abutment 204 prevents the absorbent fiber block 225 from dislodging from the cap 200.

FIG. 6 shows a plug 220. The plug 220 has a distal end 220 that is wider than an inner diameter of the distal portion of the cap. The plug 220 has a proximal portion 222 that is approximately the same diameter as an inner diameter of the distal portion of the cap 200. The proximal portion forms a snug fit when inserted in the cap 200. A proximal abutment 221 contacts a distal end of the wide portion 220 when the plug is fully inserted. The plug 240 includes a narrow wall 223 and conical portion 224 to urge the absorbent fiber block 225 toward the proximal abutment 204 when the plug 240 is inserted.

To assemble the cap 200, the cylindrical portion including the narrow wall 202 and wide outer wall 203 is fabricated, for example, by molding. Next, the absorbent fiber block 2225 is inserted from the distal end of the cylindrical portion. Then, the cap 240 is seated within the distal end of the cylindrical portion.

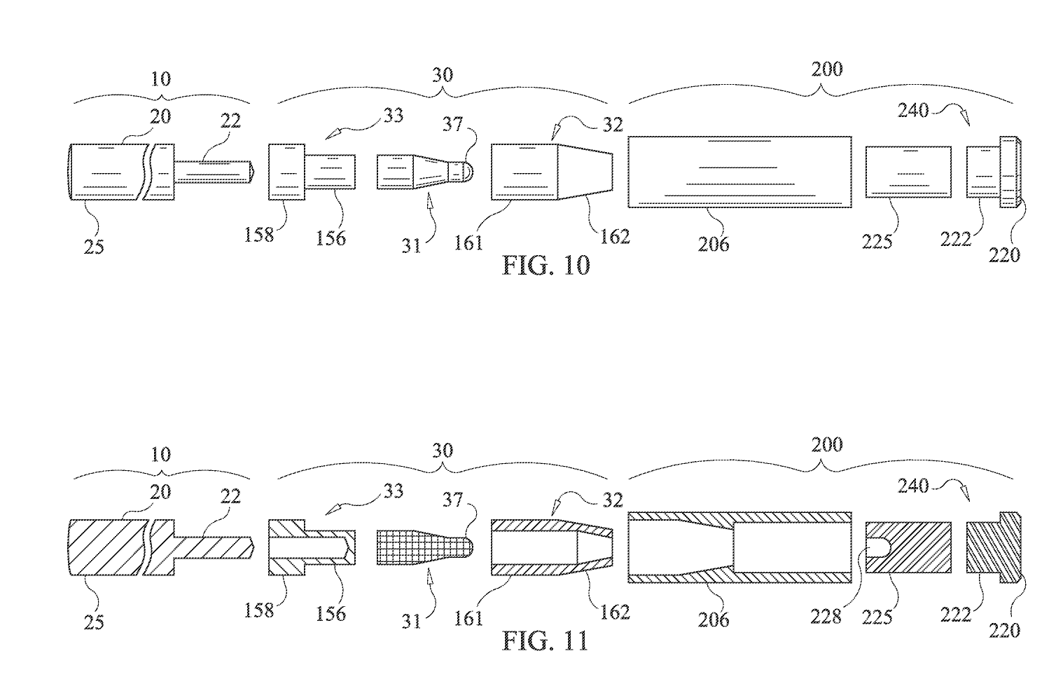

FIGS. 7-8 show a rod assembly 10, connected to a marker 30, inserted in a cap 200. FIG. 9 shows a rod assembly 10, a marker 30, and a cap 200 aligned axially for connection with each other. FIGS. 10 and 11 show the rod assembly, the marker 30, and the cap in exploded and exploded sectional views.

A preferred method of using the marker 30 shown in FIGS. 7-9 includes a method marking tissue in a laparoscopic surgical procedure. In a first step of the method, a peg 22 of a rod assembly 10 is inserted into the socket 34 of the connector 33. When the marker 30 is ready to be inserted, the surgeon removes the cap 200 from the marker 30. Next, the surgeon delivers the marker 30 to the targeted tissue by pushing the marker 30 through a cannula to the targeted area. The surgeon then manipulates and maneuvers the marker 30 to the targeted tissue by operating the proximal end 25 of the rod 20. The tip 37 of the marker 30 is pressed against the targeted tissue to leave an inked mark on the targeted tissue.

In a further embodiment of method of using the marker 30, the marker 30 can be used during the installation of a diaphragmatic pacemaker. The surgeon begins by making an incision and inserting a cannula and an endoscope. The surgeon inserts the marker by pushing the marker 30 through the cannula with the rod 20. The surgeon examines an inferior surface of the diaphragm and locates the phrenic nerve. Portions of the phrenic nerve will respond to electric stimuli better than other portions. When a responsive portion is found, the surgeon marks that portion with the tip 37 of the marker 30. After marking the nerve, the marker 30 is removed by retracting the rod 20 from the cannula. The surgeon then connects the lead of the pacemaker to the marked portion of the phrenic nerve.

While the embodiments show preferred devices and methods, the scope of the invention may be broader than those examples.

What is claimed is:

1. A surgical marker for marking tissue laparoscopically, comprising:
a connector having a socket formed therein for receiving a peg of a rod; and
a nib for holding and dispensing ink on tissue, said nib being connected to said proximal connector.

2. The surgical marker according to claim 1, wherein said nib is made from an absorbent material.

3. The surgical marker according to claim 1, further comprising a holder for securing said nib to said connector, said holder having a hole formed therein, said holder being configured to allow said nib to extend from said hole when said holder is fastened to said connector.

4. The surgical marker according to claim 3, wherein:
said connector has a proximal wider portion, a distal narrower portion with a diameter, and an abutment between said proximal wider portion and said distal narrower portion;
said holder has a passage with a diameter for receiving said nip, said passage being connected to said hole; and
said diameter of said passage being at least as wide as said diameter of said distal narrower portion to allow said holder to fit over said distal narrower portion.

5. The marker according to claim 4, wherein said diameter of said passage is substantially equal to said diameter of said distal narrower portion so as to form a snug fit.

6. The marker according to claim 4, wherein said diameter of said passage is not larger than a diameter of said proximal wider portion.

7. The marker according to claim 1, wherein said socket has a length, said length of said socket being at least as long as a length of the peg.

8. The marker according to claim 1, wherein said socket has a diameter, said diameter of said socket being at least as wide as a diameter of the peg.

9. The marker according to claim 1, wherein:
said socket has a length, said length of said socket being at least as long as a length of the peg;
said socket has a diameter, said diameter of said socket being at least as wide as a diameter of the peg; and
said socket is cylindrical shaped and forms a snug fit and connection with the rod when said socket receives the peg.

10. The marker according to claim 3, wherein:
said holder has a passage with a diameter for receiving said nip, said passage being connected to said hole; and
said passage of said holder has a frustoconical portion, said frustoconical portion contacting said marker when said holder is secured to said connector.

11. The marker according to claim 1, wherein said marker has a width less than an inner diameter of a trocar to be used laparoscopically.

12. The marker according to claim 1, further comprising a rod having a peg, said peg being inserted in said socket of said connector, said rod being configured to deliver said marker to tissue within a patient.

13. The marker according to claim 12, wherein said marker is no wider than said rod.

14. A cap for storing ink of a marker, comprising:
an enclosure with an opening formed therein, said opening being configured to receive the marker and to form an air-tight closure with the marker when said enclosure receives the marker; and
an absorbent fiber block for holding ink, said absorbent fiber block being disposed in said enclosure, said block contacting the marker when said enclosure receives the marker to allow the ink to diffuse from said absorbent fiber block to the marker.

15. The cap according to claim 14, wherein said absorbent fiber block has a dimple formed therein, said dimple being configured to receive a tip of a nib of the marker when said enclosure receives the marker.

16. The cap according to claim 14, wherein said enclosure is cylindrical shaped.

17. The cap according to claim 14, wherein said opening of said enclosure has an inner proximal abutment, said inner proximal abutment narrowing said opening to a width narrower than a width of said absorbent fiber block, said inner proximal abutment securing said absorbent fiber block within said enclosure.

18. A method for assembling a surgical marker, which comprises:
providing a surgical marker according to claim 1;
providing a rod having a distal peg; and
inserting said distal peg into said socket of said connector.

19. A method for marking tissue in a laparoscopic procedure, which comprises:
observing a target location in the patient with a laparoscope;
inserting a marker according to claim 1 to the target location by pushing said marker with a rod through a cannula;
identifying the target tissue by viewing the target tissue with said laparoscope;
marking the target tissue with said marker while observing the target tissue with the laparoscope to create marked target tissue.

20. The method according to claim 19, which further comprises connecting a pacemaker lead to the marked target tissue while observing said marked target tissue with said laparoscope.
